(19) United States
(12) Patent Application Publication
(10) Pub. No.: US 2022/0400719 A1
(43) Pub. Date: Dec. 22, 2022

(54) METHOD FOR DISPERSING A SEAWEED POWDER IN WATER

(71) Applicant: Cargill, Incorporated, Wayzata, MN (US)

(72) Inventors: Guèba AGODA-TANDJAWA, Bretteville-sur-On (FR); Cindy Le Garnec, Le Dezert (FR)

(73) Assignee: Cargill, Incorporated, Wayzata, MN (US)

(21) Appl. No.: 17/762,808

(22) PCT Filed: Sep. 23, 2020
(86) PCT No.: PCT/US2020/052162

§ 371 Date: Mar. 23, 2022

(30) Foreign Application Priority Data
Sep. 23, 2019 (EP) 19198948.2

(51) Int. Cl. A23L 17/60; A23L 2/52; A23L 17/00

(52) U.S. Cl. CPC A23L 17/60; A23L 2/52; A23L 17/70

(57) ABSTRACT

The present invention relates to a method of dispersing a seaweed powder in an aqueous environment, comprising the steps of (a) providing a seaweed powder and an aqueous environment; and (b) dispersing the seaweed powder in the aqueous environment at a pH of at least 3.5, preferably at most 9.0. The invention further relates to a dispersion of seaweed powder in an aqueous environment obtained by the method of the invention and its use in food, beverages, nutritional products, dietary supplements, feed, personal care applications, pharmaceutical applications and industrial applications.

### FIELD OF THE INVENTION

The present invention relates to a method for dispersing a seaweed powder in an aqueous environment. The invention further relates to a dispersion of seaweed powder in an aqueous environment obtained by the method of the invention and its use in food, beverages, nutritional products, dietary supplements, feed, personal care applications, pharmaceutical applications and industrial applications.

### BACKGROUND OF THE INVENTION

It is believed that the amount of seaweed production in the world is in the order of 20,000,000 t/year. Recently, improved ways of cultivating and harvesting of seaweeds were developed not only to increase production but also to enable a more efficient growth control. EP 2 230 895, EP 3 246 292 and WO 2017/131510 disclose examples of a cultivating system of seaweeds.

Seaweeds are plant-like organisms that generally live attached to rock or other hard substrata in marine environments. Seaweeds may be microscopic such as microalgae but also enormous such as giant kelp that grows in “forests” and tower like underwater woods from their holdfasts at the bottom of the sea. Most of the seaweed species are either green (more than 6500 species), brown (about 2000 species), or red (about 7000 species) kinds.

Since hundreds of years, people recognized that seaweeds are beneficial for human as well as animal health and recently, various studies demonstrated that seaweeds are effective as fat substitutes. As people become more aware of the relation between diet and health, the consumption of seaweeds has been and is increasingly gaining attention. Nowadays, many new food products based on seaweeds have been developed and marketed, offering enhanced health benefits and the potential to decrease the risk of diseases. In addition to the vast health benefits when consumed directly or after minor pre-processing as dietary supplements, the seaweeds have a range of natural functional properties such as nutritional, physicochemical and textural properties; and when used as ingredients to manufacture various products, seaweeds may transfer their advantageous functional properties thereto.

It is however difficult to disperse a dry seaweed powder in an efficient and economical manner while preserving their functional properties. Typically, one needs to use longer dispersing times and/or higher shear forces in order to achieve a good dispersion having optimal rheological properties. There is therefore a need for an efficient and economical method of dispersing a functional seaweed powder, i.e. a seaweed powder having functional properties, in an aqueous environment while ensuring that the resulting dispersion benefits optimally from the rheological properties of the seaweed.

### SUMMARY OF THE INVENTION

The present invention provides a method of dispersing a dry seaweed powder in an aqueous environment. The present inventors observed that the method according to the invention (hereinafter “the inventive method”) is able to produce a seaweed dispersion in an efficient and economical manner while ensuring that said dispersion has optimum rheological properties. They also observed that the obtained dispersion has advantageous properties. When used in food products for example, said dispersion may positively influence the texture, flow, mouthfeel and/or ingestion of said products. When used in personal care products, said dispersion may positively influence the appearance of the product and allow for an optimum transfer of active materials present in such products to hair, skin or other places in need of care. The same may be true for pharmaceutical products also.

Other advantages of the inventive dispersion will become apparent from the detailed description of the invention given hereunder.

### EXPLANATION OF THE FIGURES

FIG. 1 shows the methodology to determine the C₀ of a seaweed-based powder sample.

### DETAILED DESCRIPTION OF THE INVENTION

The invention relates to a method (hereinafter the inventive method) of dispersing a seaweed powder in an aqueous environment, comprising the steps of:
a. Providing a seaweed powder and an aqueous environment, the seaweed powder having a storage modulus (G′) of at least 10 Pa as determined on a 0.3 wt % aqueous dispersion of said powder;
b. Dispersing the seaweed powder in the aqueous environment at a pH of at least 3.5, preferably at most 9.0.

By seaweed powder is herein understood a collection of seaweed particles, i.e. said powder contains seaweed particles. Said particles may be obtained by crushing or milling a seaweed in wet or dry form. The most preferred seaweed powder utilized in the present invention is a powder obtained according to the methods described in patent applications EP19164267.7 and EP19195710.9, both applications being incorporated herein in their entirety by reference, since such powders have excellent rheological properties, e.g. a combination of high elastic modulus (G′) and low critical gelling concentration (C₀).

Preferably, the seaweed particles have a D50 of preferably at least 20 μm, more preferably at least 50 μm, even more preferably at least 75 μm, even more preferably at least 85 μm, most preferably at least 120 μm. Preferably, said DSO is at most 750 μm, more preferably at most 500 μm, even more preferably at most 350 μm, most preferably at most 250 μm. Preferably, said DSO is between 20 μm and 750 μm, more preferably between 50 μm and 350 μm, most preferably between 75 μm and 250 μm.

Preferably, the seaweed particles have a D90 of preferably at least 125 μm, more preferably at least 100 μm, even more preferably at least 175 μm, most preferably at least 220 μm. Preferably, said D90 is at most 800 μm, more preferably at most 600 μm, most preferably at most 400 μm. Preferably, said D90 is between 125 μm and 800 μm, more preferably between 175 μm and 600 μm, most preferably between 220 μm and 400 μm.

Preferably, the seaweed particles have a DSO of at least 20 μm and a D90 of at least 125 μm, more preferably a DSO of at least 50 μm and a D90 of at least 175 μm, most preferably a DSO of at least 75 μm and a D90 of at least 220 μm.

Preferably, the seaweed powder utilized in the inventive composition contains at least 80% dry basis of seaweed particles, more preferably at least 90% dry basis, even more preferably at least 92% dry basis, most preferably at least 96% wt % dry basis. The remaining wt % up to 100 wt % may contain foreign materials other than the seaweed particles which formed part of the biomass, e.g. algae, other strains of seaweed, etc.

The seaweed suitable for the present invention may be selected from numerous types of seaweeds. In the present context by “seaweed” is understood a macroscopic, multicellular, marine algae which can grow in the wild or can be farmed. Wild seaweeds typically grow in the benthic region of the sea or ocean without cultivation or care from humans. Farmed seaweeds are typically cultivated on various supports like ropes, fabrics, nets, tube-nets, etc., which are typically placed below the surface of the sea or ocean. Seaweeds may also be farmed in pools, ponds, tanks or reactors containing seawater and placed on the shore or inland. The term “seaweed” includes members of the red, brown and green seaweeds.

Throughout this document, certain taxonomies of seaweeds' families, genera, etc. are used. The referred taxonomies are those typically used in the art of seaweed cultivation and harvesting and/or in the art of seaweed extracts. An explanation of the taxonomies of red seaweeds are for example given by C. W. Schneider and M. J. Wynne in Botanica Marina 50 (2007): 197-249; by G. W. Sauders and M. H. Hommersand in American Journal of Botany 91(10): 1494-1507, 2004; and by Athanasiadis, A. in Bocconea 16(1): 193-198.2003.—ISSN 1120-4060. An explanation of the taxonomies of green seaweeds is for example given by Naselli-Flores L and Barone R. (2009) Green Algae. In: Gene E. Likens, (Editor) Encyclopedia of Inland Waters. volume 1, pp. 166-173 Oxford: Elsevier. An explanation of the taxonomies of brown seaweeds is for example given by John D. Wehr in Freshwater Algae of North America—Ecology and Classification, Edition: 1, Chapter: 22, Publisher: Academic Press, Editors: John D. Wehr, Robert G. Sheath, pp. 757-773.

Preferably, the seaweed used in accordance with the invention is a red seaweed, i.e. a seaweed belonging to Rhodophyta phylum; or a brown seaweed, i.e. orders, families and genera in the class Phaeophycaeae. Red seaweeds have a characteristic red or purplish colour imparted by pigments present in the seaweed and called phycobilin, e.g. phycoerythrin.

More preferably, the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof. Most preferably, the seaweed is selected from the genera of Bangiales, Chondrus, Iridaea, Palmaria, Gigartina, Gracilaria, Gelidium, Rhodoglossum, Hypnea, Eucheuma, Kappaphycus, Agarchiella, Gymnogongrus, Sarcothalia, Phyllophora, Ahnfeltia, Mazzaella, Mastocarpus, Chondracanthus, Furcellaria and mixtures thereof. Best results were obtained when the seaweed was chosen from the group of seaweeds consisting of Porphyra sp., Palmaria palmata, Eucheuma spinosum, Eucheuma denticulatum, Eucheuma sp., Eucheuma cottonii (also known as Kappaphycus alvarezii), Kappaphycus striatus, Kappaphycus sp., Chondrus crispus, fish moss, Fucus crispus, Chondrus sp, Sarcothalia crispata, Mazzaella laminaroides, Mazzaella sp., Chondracanthus acicularis, Chondracanthus chamissoi, Chondracanthus sp., Gigartina pistilla, Gigartina mammillosa, Gigartina skottsbergii, Gigartina sp., Gracilaria sp, Gelidium sp., Mastocarpus stellatus and mixtures thereof.

It is known that some of the red seaweeds, e.g. Kappaphycus alvarezii, may have green or brown strains; however, within the context of the present invention when mentioning for example that the seaweed is a red seaweed, it is herein meant the phylum and not the colour of the strains.

Most preferred brown seaweeds are those chosen from the families Acsophyllum, Durvillaea, Ecklonia, Hyperborea, Laminaria, Lessonia, Macrocystis, Fucus and Sargassum. Specific examples of brown seaweeds include Bull Kelp (Durvillae potatorum), Durvillae species, D. antarctica and Knotted Kelp (Ascophyllum nosodum).

The seaweed powder has a storage modulus (G′) of at least 10 Pa as determined on a 0.3 wt % aqueous dispersion of said powder. Preferably, said powder has a critical gelling concentration (C₀) of at most 0.5 wt %, more preferably at most 0.3 wt %, most preferably at most 0.1 wt %. Preferably, said powder has a G′ of at least 15 Pa, more preferably at least 20 Pa, more preferably at least 30 Pa, more preferably at least 50 Pa, more preferably at least 70 Pa, more preferably at least 90 Pa, even more preferably at least 110 Pa, most preferably at least 120 Pa. Preferably, said G′ is at most 500 Pa, more preferably at most 400 Pa, even more preferably at most 300 Pa, most preferably at most 200 Pa.

Preferably, the seaweed powder has a storage modulus (G′) of at least 10 Pa as determined on a 0.3 wt % aqueous dispersion of said powder and a critical gelling concentration (C₀) of at most 0.5 wt %, wherein the seaweed is a red seaweed, i.e. a seaweed belonging to Rhodophyta phylum. Preferred ranges of the G′ and C₀ are given above and will not be repeated herein. Preferably, said powder has a CIELAB L* value of at least 50, preferably at least 60, preferably at least 70, preferably at least 74, more preferably at least 76, even more preferably at least 78, most preferably at least 80. Preferably, the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof. Most preferably, the seaweed is selected from the genera of Bangiales, Chondrus, Iridaea, Palmaria, Gigartina, Gracilaria, Gelidium, Rhodoglossum, Hypnea, Eucheuma, Kappaphycus, Agarchiella, Gymnogongrus, Sarcothalia, Phyllophora, Ahnfeltia, Mazzaella, Mastocarpus, Chondracanthus, Furcellaria and mixtures thereof.

Most preferably, the seaweed powder has a storage modulus (G′) of at least 10 Pa as determined on a 0.3 wt % aqueous dispersion of said powder and a critical gelling concentration (C₀) of at most 0.5 wt %, wherein the seaweed is a red seaweed chosen from the group consisting of Eucheuma spinosum, Eucheuma Cottonii (Kappaphycus alvarezii), Chondrus crispus and combinations thereof. Preferred ranges of the G′ and C₀ are given above and will not be repeated herein. Preferably, said powder has a CIELAB L* value of at least 50, preferably at least 60, preferably at least 70, preferably at least 74, more preferably at least 76, even more preferably at least 78, most preferably at least 80.

Preferably, the seaweed powder contains an amount of acid insoluble material (AIM) of at most 50 wt % relative to the weight of the powder, more preferably at most 40 wt %, even more preferably at most 30 wt %, most preferably at most 20 wt %. Preferably, said AIM content is at least 1 wt %, more preferably at least 5 wt %, most preferably at least 10 wt %. It was observed that when the seaweed powder has an AIM content within the preferred ranges, it's nutritional properties were optimized.

Preferably, the seaweed powder contains an amount of acid insoluble ashes (AIA) of at most 5.0 wt % relative to the weight of the powder, more preferably at most 3.0 wt %, even more preferably at most 1.0 wt %, most preferably at most 0.80 wt %. Preferably, said AIA content is at least 0.01 wt %, more preferably at least 0.05 wt %, most preferably at least 0.10 wt %. It was observed that a seaweed powder having an AIA content within the preferred ranges, is more suitable for use in food, personal care and pharmaceutical products as it does not introduce, or introduce to a lesser extent, foreign materials into said products, which in turn may require additional purification steps of said products.

Preferably, the seaweed powder has a storage modulus (G′) of at least 10 Pa as determined on a 0.3 wt % aqueous dispersion of said powder, a critical gelling concentration (C₀) of at most 0.5 wt % and a cadmium content of at most 1.1 ppm, wherein the seaweed is a red seaweed chosen from the group of seaweeds consisting of Porphyra sp., Palmaria palmata, Eucheuma spinosum, Eucheuma denticulatum, Eucheuma sp., Eucheuma cottonii (also known as Kappaphycus alvarezii), Kappaphycus striatus, Kappaphycus sp., Chondrus crispus, Irish moss, Fucus crispus, Chondrus sp, Sarcothalia crispata, Mazzaella laminaroides, Mazzaella sp., Chondracanthus acicularis, Chondracanthus chamissoi, Chondracanthus sp., Gigartina pistilla, Gigartina mammillosa, Gigartina skottsbergii, Gigartina sp., Gracilaria sp, Gelidium sp., Mastocarpus stellatus and mixtures thereof. Preferably, said cadmium content is at most 0.9 ppm, even more preferably at most 0.7 ppm, most preferably at most 0.5 ppm. Preferably, said powder has a G′ of at least 30 Pa, more preferably at least 50 Pa, more preferably at least 60 Pa, more preferably at least 70 Pa, more preferably at least 90 Pa, even more preferably at least 110 Pa, most preferably at least 120 Pa. Preferably, said G′ is at most 500 Pa, more preferably at most 400 Pa, even more preferably at most 300 Pa, most preferably at most 200 Pa. Preferably, the C₀ of said powder is between 0.001 and 0.100 wt %, more preferably between 0.005 and 0.090 wt %, most preferably between 0.010 and 0.080 wt %. More preferably, C₀ is between 0.001 and 0.080 wt %, more preferably between 0.005 and 0.060 wt %, even more preferably between 0.010 and 0.050 wt %, most preferably between 0.010 and 0.040 wt %. Preferably, said powder has a G′ of at least 40 Pa and a C₀ of between 0.001 and 0.100 wt %, more preferably between 0.005 and 0.090 wt %, most preferably between 0.010 and 0.080 wt %. Preferably, said powder has a G′ of at least 90 Pa and a C₀ of between 0.001 and 0.100 wt %, more preferably between 0.005 and 0.090 wt %, most preferably between 0.010 and 0.080 wt %. Preferably, said powder has a G′ of at least 120 Pa and a C₀ of between 0.001 and 0.100 wt %, more preferably between 0.005 and 0.090 wt %, most preferably between 0.010 and 0.080 wt %. Preferably, said powder has a CIELAB L* value of at least 50, preferably at least 60, preferably at least 70, preferably at least 74, more preferably at least 76, even more preferably at least 78, most preferably at least 80. Preferably, the seaweed is chosen from the group consisting of Eucheuma spinosum, Eucheuma cottonii (also known as Kappaphycus alvarezii), Chondrus crispus Irish moss and mixtures thereof.

The term “aqueous environment” as used herein means a liquid medium which contains water, non-limiting example thereof including pure water, a water solution and a water suspension, but also aqueous liquid mediums such as those contained by dairy products, e.g. reconstituted skimmed milk, milk, yoghurt and the like; by personal care products such as lotions, creams, ointments and the like; and pharmaceutical products. Within the context of the present invention, preferred aqueous environments are water (purified or tap water), milk and reconstituted skimmed. Preferably, the aqueous environment contains at least 30 wt % water based on the total weight of said environment, more preferably at least 40 wt % water, even more preferably at least 50 wt % water, even more preferably at least 60 wt % water, even more preferably at least 70 wt % water, even more preferably at least 80 wt % water, most preferably at least 90 wt % water. The remaining wt % up to 100% may comprise additives; preservatives; vitamins; sterols like phytosterols; antioxidants like polyphenols; beneficial minerals for human nutrition; whole vegetable extracts; cellulose such as microfibrillated cellulose and cellulose gel; dextrin; maltodextrin; sugars like sucrose, glucose; polyols like mannitol, erythritol, glycerol, sorbitol, xylitol, maltitol; protein or protein hydrolysate like plants or vegetables proteins and dairy proteins; oils and fat; surfactants; lecithin; glucomannans and/or galactomannans, e.g. guar gum, xanthan gum, locust bean gum, cassia gum, tara gum, konjac gum, alginate, agar, gellan gum, carrageenan and beta 1,3 glucan; native starch; modified starch; and combinations thereof.

Preferably, the aqueous environment contains a salt. Any salt soluble in water can be utilized, non-limiting examples including chloride salts, e.g. sodium chloride, potassium chloride, calcium chloride and ammonium chloride; sulphate salts, e.g. magnesium sulphate, iron sulphate, calcium sulphate, potassium sulphate, sodium sulphate; nitrate salts, e.g. calcium nitrate, sodium nitrate, potassium nitrate; phosphate salts, e.g. sodium phosphate, calcium phosphate, potassium phosphate; salts of organic acids and combinations thereof. Preferably, the salt is sodium chloride or potassium chloride. Most preferably, the salt used is a food grade salt, i.e. a salt as defined in the “Codex standard for food grade salt”, CX STAN 150-1985, Rev. 1-1997, Amend, 1-1999, Amend 2-2001.

Good results may be obtained when the aqueous environment has an ionic strength of at least 0.01 M, more preferably at least 0.05 M, most preferably at least 0.10 M. Preferably, said ionic strength is at most 10.00 M, more preferably at most 5.00 M, most preferably at most 3.00 M. Preferably, said ionic strength is between 0.05 and 1.00 M, more preferably between 0.10 and 0.80 M, most preferably between 0.15 and 0.60 M. The ionic strength of the aqueous environment may be adjusted by adding salts thereto, most preferred salts being sodium and calcium chloride and sodium hydroxide. If food products are intended to be manufactured by using the obtained dispersion, said salts should be food grade salts. The concentration of salts in the aqueous environment can be routinely adjusted to reach the desired ionic strength.

To ensure that the dispersion is carried out at the required pH, the aqueous environment has preferably a pH of at least 3.5, more preferably at least 4.0, more preferably at least 4.5, even more preferably at least 5.0, even more preferably at least 5.5, most preferably at least 6.0. Preferably, the pH of the aqueous environment is at most 9.0, more preferably at most 8.5, even more preferably at most 8.0, most preferably at most 7.5. Preferably, said pH is between 3.5 and 9.0, more preferably between 4.0 and 9.0, more preferably between 4.5 and 8.5, even more preferably between 5.0 and 8.5, even more preferably between 5.5 and 8.0, most preferably between 6.0 and 7.5. The pH of the aqueous environment can be adjusted by well-known means, e.g. by adding a base (or an alkali), preferably a food grade base or by using a pH buffer. A buffer solution (more precisely, pH buffer or hydrogen ion buffer) is an aqueous solution consisting of a mixture of a weak acid and its conjugate base, or vice versa. Its pH changes very little when a small amount of strong acid or base is added to it. Buffer solutions are used as a means of keeping pH at a nearly constant value in a wide variety of applications, e.g. food, personal care and pharma applications. Preferably, a food grade base is utilized to adjust the pH of the aqueous environment, non-limiting examples thereof including ammonium hydroxide or aqueous ammonia, sodium hydroxide, sodium bicarbonate, potassium hydroxide, potassium carbonate and calcium hydroxide, quicklime/calcium oxide, calcium carbonate, and mixtures thereof. The pH can be measured with any pH-meter known in the art after carrying out its calibration (if required) and using it as indicated in the operating instructions.

Dispersing the seaweed powder in the aqueous environment can be carried out by any known meaning in the art. Suitable techniques include shear treatments and high shear treatments, pressure homogenization, cavitation, explosion, pressure increase and pressure drop treatments, colloidal milling, blending, extrusion, ultrasonic treatment, and combinations thereof. Advantageously, simple mixing devices can be used, such as high shear mixers (e.g. of the ULTRA TURRAX type) but also low shear mixers such as for example, magnetic stirrers or mechanical stirrers, e.g. an IKA® Eurostar mechanical stirrer equipped with an R1342 4-bladed propeller stirrer or a Silverson L4RT overhead batch mixer equipped with an Emulsor Screen (e.g. with round holes of about 1 mm diameter) or various mixers using an IKA (RWD 20) with 4-bladed propeller set at between 100 and 1500 rpm.

Preferably, the seaweed powder is dispersed in the aqueous environment in an amount of at least 0.1 wt % based on the total dry solids content of said environment, more preferably at least 0.3 wt %, most preferably at least 0.5 wt %. Preferably, said amount is at most 50 wt %, more preferably at most 30 wt %, most preferably at most 10 wt %. As used herein, the term “dry solids” (DS) means the ratio of the weight of the solid content contained by a sample and the total weight of said sample. The solid content is herein understood the content of a sample obtained by evaporating the water contained by said sample by drying 5 g of the sample for 4 hours at 120° C. under vacuum (e.g. below 0.5 bar).

The dispersion of the seaweed powder in the aqueous environment is carried out at a pH of at least 3.5. Preferably, said pH is at least 4.0, more preferably at least 4.5, even more preferably at least 5.0, even more preferably at least 5.5, most preferably at least 6.0. Preferably, the pH of the aqueous environment is at most 9.0, more preferably at most 8.5, even more preferably at most 8.0, most preferably at most 7.5. Preferably, said pH is between 3.5 and 9.0, more preferably between 4.0 and 9.0, more preferably between 4.5 and 8.5, even more preferably between 5.0 and 8.5, even more preferably between 5.5 and 8.0, most preferably between 6.0 and 7.5. The simplest way to ensure that the dispersion is carried out at the required pH is to adjust the pH of the aqueous environment as already indicated hereinabove. Therefore, preferably, the inventive method comprises the steps of:
c. Providing a seaweed powder and an aqueous environment having a pH of at least 3.5;
d. Dispersing the seaweed powder in the aqueous environment while maintaining the pH essentially constant.

The pH can be maintain essentially constant during the dispersion of the seaweed in the aqueous environment by utilizing a pH buffer solution or by monitoring the pH during dispersion and adjusting it with for example a base as exemplified above.

The inventive method provides an aqueous dispersion of the seaweed in the aqueous environment. By “aqueous dispersion” is herein understood a composition wherein said powder is dispersed in the aqueous environment, which forms a continuous phase. The powder may be dispersed inside the aqueous environment (i.e. in the bulk) but can also be present at any interface present in said aqueous environment, e.g. the interface between water and any component other than the powder, e.g. oil. Examples of dispersions include without limitation suspensions, emulsions, solutions and the like.

Preferably, the obtained dispersion is a suspension or an emulsion. In case it is desired to obtain an emulsion, an oil phase is added before, during or after dispersing the seaweed in the aqueous environment and a shear treatment is applied on the obtained composition to create the emulsion. Processes for manufacturing the emulsion are widely known in the art. Preferably, an emulsifier is utilized to facilitate the formation of the emulsion, non-limiting examples thereof including mono and diglycerides; distilled monoglycerides; mono- and diglycerides of saturated or unsaturated fatty esters; diacetyl tartaric acid esters of mono- and diglycerides (DATEM); modified lecithin; polysorbate 20, 40, 60 or 80; sodium stearyl lactylate; propylene glycol monostearate; succinylated mono- and diglycerides; acetylated mono- and diglycerides; propylene glycol mono- and diesters of fatty acids; polyglycerol esters of fatty acids; lactylic esters of fatty acids; glyceryl monosterate; propylene glycol monopalmitate; glycerol lactopalmitate and glycerol lactostearate; lecithin; and mixtures thereof. The emulsifiers may be used independently, or two or more kinds may be used in combination.

Preferably, the inventive method includes an emulsification step wherein the dispersion obtained at step b) is used to prepare an emulsion, preferably an oil-in-water emulsion. The oil-in-water emulsion is preferably an edible emulsion. The edible oil-in-water emulsion preferably comprises from 5 to 80 wt-% of oil. The oil typically is an edible oil. As understood by the skilled person such edible oils typically comprise triglycerides, usually mixtures of such triglycerides. Typical examples of edible oils include vegetable oils including palm oil, rapeseed oil, linseed oil, sunflower oil and oils of animal origin.

The inventive method may also be utilized to prepare emulsions in the form of a dressing or a similar condiment. Preferably, the edible dressing comprises from 15 to 72 wt-% of oil. It is particularly preferred that the composition in the form of an oil-in-water emulsion is a mayonnaise or a spread.

The inventive method may also be utilized to prepare emulsified products comprising proteins. Thus, the inventive method preferably includes an emulsification step wherein the dispersion obtained at step b) is used to prepare an emulsion, preferably an oil-in-water emulsion, comprising protein, wherein the amount of protein is preferably from 0.1 to 10 wt %, more preferably from 0.2 to 7 wt % and even more preferably from 0.25 to 4 wt % by weight of the emulsion.

Preferably, the dispersion of the seaweed in the aqueous environment in step b) takes place at a dispersing temperature of between 10° C. and 40° C., more preferably between 15° C. and 30° C.

Preferably, the dispersion of the seaweed in the aqueous environment is carried out for a dispersing time of at least 5 minutes, more preferably at least 10 min, even more preferably at least 15 min, most preferably at least 20 min. Preferably, the dispersing time is at most 60 min, more preferably at most 55 min, even more preferably at most 50 min, most preferably at most 45 min. Preferably, the dispersing time is between 5 and 55 min, more preferably between 10 and 50 min, even more preferably between 15 and 45 min, most preferably between 20 and 40 min.

After being dispersed, the dispersion obtained at step b) is heated to a temperature of at least 20° C., more preferably at least 40° C., even more preferably at least 60° C., most preferably at least 80° C. Preferably, said temperature is at most 95° C., more preferably at most 93° C., even more preferably at most 91° C., most preferably at most 90° C. Preferably, said temperature is between 20 and 95° C., more preferably between 40 and 93° C., even more preferably between 60 and 91° C., most preferably between 80 and 90° C. Preferably, said dispersion is heated under stirring. Preferably, said dispersion is kept at said temperature for a heating time of at least 5 minutes, more preferably at least 10 min, even more preferably at least 15 min, most preferably at least 20 min. Preferably, the heating time is at most 60 min, more preferably at most 55 min, even more preferably at most 50 min, most preferably at most 45 min Preferably, the heating time is between 5 and 55 min, more preferably between 10 and 50 min, even more preferably between 15 and 45 min, most preferably between 20 and 40 min.

The invention further relates to a dispersion (hereinafter the inventive dispersion) of a seaweed in an aqueous environment, said dispersion having a pH of at least 3.5. Preferred embodiments of the seaweed and seaweed amount, of the aqueous environment and of the pH are given hereinabove and will not be repeated. Preferably, said dispersion has an ionic strength of at least 0.01 M, more preferably at least 0.05 M, most preferably at least 0.10 M. Preferably, said ionic strength is at most 10.00 M, more preferably at most 5.00 M, most preferably at most 3.00 M. Preferably, said ionic strength is between 0.05 and 1.00 M, more preferably between 0.10 and 0.80 M, most preferably between 0.15 and 0.60 M.

Preferably, the inventive dispersion has a pH of at least 4.0, more preferably at least 4.5, even more preferably at least 5.0, even more preferably at least 5.5, most preferably at least 6.0 and an ionic strength of at least 0.01 M. Preferably, the inventive dispersion has a pH of at least 4.0, more preferably at least 4.5, even more preferably at least 5.0, even more preferably at least 5.5, most preferably at least 6.0 and an ionic strength of at least 0.05 M. Preferably, the inventive dispersion has a pH of at least 4.0, more preferably at least 4.5, even more preferably at least 5.0, even more preferably at least 5.5, most preferably at least 6.0 and an ionic strength of at least 0.10 M. Preferably, said pH is between 3.5 and 9.0, more preferably between 4.0 and 9.0, more preferably between 4.5 and 8.5, even more preferably between 5.0 and 8.5, even more preferably between 5.5 and 8.0, most preferably between 6.0 and 7.5. Preferably, said ionic strength is at most 10.00 M, more preferably at most 5.00 M, most preferably at most 3.00 M. Preferably, said ionic strength is between 0.05 and 1.00 M, more preferably between 0.10 and 0.80 M, most preferably between 0.15 and 0.60 M.

The invention further relates to a dispersion obtainable by the method of the invention.

The invention also relates to a dispersion of a seaweed in an aqueous environment, said dispersion having a pH of at least 4.0 and an elastic modulus (G′) of at least 20 Pa, preferably at least 30 Pa, more preferably at least 40 Pa, even more preferably at least 50 Pa, even more preferably at least 60 Pa, most preferably at least 70 Pa. Preferably, said dispersion has a pH of at least 4.5 and an elastic modulus (G′) of at least 20 Pa, preferably at least 30 Pa, more preferably at least 40 Pa, even more preferably at least 50 Pa, even more preferably at least 60 Pa, even more preferably at least 70 Pa, most preferably at least 80 Pa. Preferably, said dispersion has a pH of at least 6.0 and an elastic modulus (G′) of at least 20 Pa, preferably at least 30 Pa, more preferably at least 40 Pa, even more preferably at least 50 Pa, even more preferably at least 60 Pa, even more preferably at least 70 Pa, most preferably at least 80 Pa. Preferably, said dispersion has a pH of between 4.0 and 8.0 and an elastic modulus (G′) of at least 20 Pa, preferably at least 30 Pa, more preferably at least 40 Pa, even more preferably at least 50 Pa, even more preferably at least 60 Pa, even more preferably at least 70 Pa, most preferably at least 80 Pa. Preferably, said G′ is at most 350 Pa, more preferably at most 250 Pa, most preferably at most 150 Pa. Preferred embodiments of the seaweed and seaweed amount and of the aqueous environment are given hereinabove and will not be repeated. Preferably, said dispersion has an ionic strength of at least 0.01 M, more preferably at least 0.05 M, most preferably at least 0.10 M. Preferably, said ionic strength is at most 10.00 M, more preferably at most 5.00 M, most preferably at most 3.00 M. Preferably, said ionic strength is between 0.05 and 1.00 M, more preferably between 0.10 and 0.80 M, most preferably between 0.15 and 0.60 M.

The invention also relates to a dispersion of a seaweed in an aqueous environment, said dispersion having a pH of at least 4.0, a tan δ of at most 0.050 and a G′ of at least 20 Pa. Preferably, said dispersion has a pH of at least 4.5, a tan δ of at most 0.040 and a G′ of at least 20 Pa. Preferably, said dispersion has a pH of at least 6.0, a tan δ of at most 0.035 and a G′ of at least 20 Pa. Preferably, said G′ is at least 30 Pa, more preferably at least 40 Pa, even more preferably at least 50 Pa, even more preferably at least 60 Pa, most preferably at least 70 Pa. Preferably, said tan δ is at most 0.035, most preferably at most 0.030. Preferably, said G′ is at most 350 Pa, more preferably at most 250 Pa, most preferably at most 150 Pa. Preferred embodiments of the seaweed and seaweed amount and of the aqueous environment are given hereinabove and will not be repeated. Preferably, said dispersion has an ionic strength of at least 0.01 M, more preferably at least 0.05 M, most preferably at least 0.10 M. Preferably, said ionic strength is at most 10.00 M, more preferably at most 5.00 M, most preferably at most 3.00 M. Preferably, said ionic strength is between 0.05 and 1.00 M, more preferably between 0.10 and 0.80 M, most preferably between 0.15 and 0.60 M.

The invention further relates to a food or a feed product containing the inventive dispersion and a nutrient. Without being bound to any theory, the inventors believe that the properties of said food or feed product are positively influenced by the advantageous properties of the inventive dispersion. In particular the inventive dispersion may enable an optimization of the transport, diffusion, and dissolution phenomena relevant to food functionalities (nutritional, sensory, and physicochemical). Moreover, said products may be easily designed to have specific flow behaviors, textures and appearances. Thus, the ability of the inventive dispersion to optimize said food functionalities may be highly beneficial for the design of food structure, which together with the classic needs (e.g. texture and mouthfeel), may enhance the impact upon wellness and health, including modulated digestion to trigger different physiological responses.

The inventive dispersion is highly suitable for use in the production of a large variety of food compositions. Examples of food compositions comprising or being manufactured by using thereof, to which the invention relates, include: luxury drinks, such as coffee, black tea, powdered green tea, cocoa, adzuki-bean soup, juice, soya-bean juice, etc.; milk component-containing drinks, such as raw milk, processed milk, lactic acid beverages, etc.; a variety of drinks including nutrition-enriched drinks, such as calcium-fortified drinks and the like and dietary fiber-containing drinks, etc.; dairy products, such as butter, cheese, yogurt, coffee whitener, whipping cream, custard cream, custard pudding, etc.; iced products such as ice cream, soft cream, lacto-ice, ice milk, sherbet, frozen yogurt, etc.; processed fat food products, such as mayonnaise, margarine, spread, shortening, etc.; soups; stews; seasonings such as sauce, TARE, (seasoning sauce), dressings, etc.; a variety of paste condiments represented by kneaded mustard; a variety of fillings typified by jam and flour paste; a variety or gel or paste-like food products including red bean-jam, jelly, and foods for swallowing impaired people; food products containing cereals as the main component, such as bread, noodles, pasta, pizza pie, corn flake, etc.; Japanese, US and European cakes, such as candy, cookie, biscuit, hot cake, chocolate, rice cake, etc.; kneaded marine products represented by a boiled fish cake, a fish cake, etc.; live-stock products represented by ham, sausage, hamburger steak, etc.; daily dishes such as cream croquette, paste for Chinese foods, gratin, dumpling, etc.; foods of delicate flavor, such as salted fish guts, a vegetable pickled in sake lee, etc.; liquid diets such as tube feeding liquid food, etc.; supplements; and pet foods. These food products are all encompassed within the present invention, regardless of any difference in their forms and processing operation at the time of preparation, as seen in retort foods, frozen foods, microwave foods, etc.

The invention also relates to an edible composition comprising the inventive dispersion, which optionally comprises an oil-based constituent. Said edible composition preferably comprises a flavor base, from 0.001 wt-% to 5 wt-% of oil, more preferably from 0.01 wt-% to 2 wt-%, even more preferably from 0.05 wt-% to 1 wt-% and even more preferably from 0.1 wt-% to 0.5 wt-% of oil with respect to the weight of the composition and an aqueous phase comprising the inventive dispersion. Herein, “flavor base” means the base of the edible composition that is responsible for the identification of the product. The flavor base preferably is a fruit- or vegetable-based product, or a mixture thereof. The edible composition is preferably a tomato-based product. Therefore, more preferably the flavor base is a tomato paste, a tomato puree, a tomato juice, a tomato concentrate or a combination thereof, and even more preferably it is a tomato paste.

The invention further relates to an oil-in-water emulsion comprising an aqueous phase containing a seaweed powder and a protein dispersed in an aqueous environment and an oil phase containing an oil, preferably a vegetable oil, wherein the amount of protein is preferably from 0.1 to 10 wt %, more preferably from 0.2 to 7 wt % and even more preferably from 0.25 to 4 wt % by weight of the emulsion. The protein may advantageously include milk protein, which is a desirable component in many food compositions. Thus, the protein preferably comprises at least 50 wt % milk protein, more preferably at least 70 wt %, even more preferably at least 90 wt % and still more preferably consists essentially of milk protein. Preferably, the emulsion is a ready-to-drink beverage, more preferably a ready-to-drink tea-based beverage. The term “ready-to-drink (tea) beverage” refers to a packaged (tea-based) beverage, i.e. a substantially aqueous drinkable composition suitable for human consumption. Preferably the beverage comprises at least 85% water by weight of the beverage, more preferably at least 90%. Ready-to-drink (RTD) milk tea beverages usually contain milk solids like for example milk protein and milk fat that give the beverages certain organoleptic properties like for example a ‘creamy mouthfeel’. Such a RTD milk tea beverage preferably comprises at least 0.01 wt % tea solids on total weight of the beverage. More preferably the beverage comprises from 0.04 to 3 wt % tea solids, even more preferably from 0.06 to 2%, still more preferably from 0.08 to 1 wt % and still even more preferably from 0.1 to 0.5 wt %. The tea solids may be black tea solids, green tea solids or a combination thereof. The term “tea solids” refers to dry material extractable from the leaves and/or stem of the plant Camellia sinensis, including for example the varieties Camellia sinensis var. sinensis and/or Camellia sinensis var. assamica. Examples of tea solids include polyphenols, caffeine and amino acids. Preferably, the tea solids are selected from black tea, green tea and combinations thereof and more preferably the tea solids are black tea solids.

The invention also relates to a product comprising the inventive dispersion and a surfactant system. Preferably, the surfactant system is in an amount of 0.1 to 50 wt-%, more preferably from 5 to 30 wt-%, and even more preferably from 10 to 25 wt-% with respect to the weight of the product. In general, the surfactants may be chosen from the surfactants described in well-known textbooks like “Surface Active Agents” Vol. 1, by Schwartz & Perry, Interscience 1949, Vol. 2 by Schwartz, Perry & Berch, Interscience 1958, and/or the current edition of “McCutcheon's Emulsifiers and Detergents” published by Manufacturing Confectioners Company or in “Tenside-Taschenbuch”, H. Stache, 2^{nd} Edn., Carl Hauser Verlag, 1981; “Handbook of Industrial Surfactants” (4^{th} Edn.) by Michael Ash and Irene Ash; Synapse Information Resources, 2008. The type of surfactant selected may depend on the type of application for which the product is intended. The surfactant system may comprise one type of surfactant, or a mixture of two or more surfactants. Synthetic surfactants preferably form a major part of the surfactant system. Thus, the surfactant system preferably comprises one or more surfactants selected from one or more of anionic surfactants, cationic surfactants, non-ionic surfactants, amphoteric surfactants and zwitterionic surfactants. More preferably, the one or more detergent surfactants are anionic, nonionic, or a combination of anionic and nonionic surfactants. Mixtures of synthetic anionic and nonionic surfactants, or a wholly anionic mixed surfactant system or admixtures of anionic surfactants, nonionic surfactants and amphoteric or zwitterionic surfactants may all be used according to the choice of the formulator for the required cleaning duty and the required dose of the cleaning composition. Preferably, the surfactant system comprises one or more anionic surfactants. More preferably, the surfactant system comprises one or more anionic surfactants selected from the group consisting of lauryl ether sulfates and linear alkylbenzene sulphonates.

For certain applications the product comprising a surfactant system preferably also comprises from 1 to 8 wt-% of an inorganic salt, preferably selected from sulfates and carbonates, more preferably selected from MgSO₄ and Na₂SO₄ and even more preferably MgSO₄. Preferably the product comprising a surfactant system is a cleaning composition, more preferably a hand dish wash composition. The product may further comprise suspended particles and/or air bubbles.

The invention further relates to a cosmetic product comprising the inventive dispersion. By cosmetic product is herein for example understood a product utilized to enhance the appearance or odor of the human or animal body. In addition to the inventive dispersion, the cosmetic product may include any further cosmetic ingredient, e.g. any ingredient commonly used in the formulation of said cosmetic products. Example of cosmetic products include skin-care creams lotions, perfumes, lipsticks, fingernail and toe nail polish, facial makeups, hair colors and hair sprays, moisturizers, gels, deodorants, hand sanitizers, baby products, bath oils, bubble baths, butters and the like. The cosmetic products of the present invention may be in any form or shape, e.g. liquid or cream emulsions.

The invention further relates to a pharmaceutical product comprising the inventive dispersion and a drug or drug releasing agent. By drug is herein understood a substance intended for use in diagnosis, cure, mitigation, treatment or prevention of a disease. The drug may be from natural origin, e.g. animal, microbial or plant origin; chemical origin, i.e. derived from chemical synthesis; or combinations thereof.

Any feature of a particular embodiment of the present invention may be utilized in any other embodiment of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the examples and comparative experiments, or where otherwise explicitly indicated, all numbers in this description indicating amounts of material or conditions of reaction, physical properties of materials and/or use are to be understood as modified by the word “about”. Unless specified otherwise, numerical ranges expressed in the format “from x to y” are understood to include x and y. When for a specific feature multiple preferred ranges are described in the format “from x to y”, it is understood that all ranges combining the different endpoints are also contemplated. For the purpose of the invention ambient (or room) temperature is defined as a temperature of about 20 degrees Celsius.

### Methods of Measurement

Ionic strength (I) and pH adjustment: the supporting dispersing liquid was standardized tap water (1.00 g/L NaCl and 0.155 g/L CaCl₂.2H₂O) of ionic strength 0.02M prepared with reverse osmosis (RO) low conductivity water (milli-Q Ultrapure Millipore 18.2MΩ·cm). The pH was adjusted with 1M NaOH and the ionic strength adjusted by spiking the required mass of salt, NaCl or CaCl₂.2H₂O. The ionic strength I of the solution (in molar concentration M) was determined according to formula:

I=0.5([A]Z_{A}²+[B]Z_{B}²+[C]Z_{C}²+ . . . )
where [A], [B], [C] are the molar concentrations of ions A, B and C and Z_{A}, Z_{B}, Z_{C} are their respective charges. See Skoog, West & Holler (1996). Fundamentals of Analytical Chemistry, 7^{th} edition (Harcourt Brace & Company, Orlando). Practically, I=c (in M) for the [1:1] electrolytes (NaCl, NaOH), I=3c for the [2:1] electrolytes (CaCl₂)).
AIM was measured by dispersing 0.5 g of sample (Wₛₐₘₚₗₑ) in 150 ml osmosis water in a 250 mL beaker. 1.5 mL of concentrated sulfuric acid were added thereto. The beaker was covered with plastic foil to prevent evaporation and heated on bain-marie at boiling temperature for 2 h. The dispersion was centrifuged at 4000 rpm (equivalent to 3250 g) for 10 minutes.
The total mass (W_{filter+dish}) of a AP 25 filter and a crystallizing dish was determined. The acidic dispersion was filtered and rinsed with osmosis water at 50° C. until its pH remained neutral (as check with a pH paper)—about 500 mL water were used. The filter with the sample was allowed to dry overnight at room temperature and further dried in an oven at 60° C. for a day and the total weight of the sample, filter and dish was determined (W_{final}) AIM (%)=[(W_{final}+W_{filter+dish})/Wₛₐₘₚₗₑ]×100.
AIA was measured as follows: 2.000 (two) grams (Wₛₐₘₚₗₑ) of sample were placed on a silica or platinum crucible, burnt for about one hour on a hot plate at 500° C. and subsequently placed in a furnace at 550° C. for 16 h. The obtained ashes were added to a solution containing 10 ml concentrated HCl and 20 ml demineralized water. The solution containing the ashes was heated to 80° C. for about half an hour and subsequently filtered using a Whatman No 40 (ash free filter). The filter containing the ashes was rinsed with water until no Cl⁻ were detected in the sample. The presence of Cl⁻ in the sample was checked with AgNO₃ (the precipitation of AgCl signifies the presence of Cl⁻).
A second silica or platinum crucible was placed in an oven at 550° C. for 10 minutes and then cooled to room temperature in a desiccator. Subsequently, the crucible was weighted (W_{crucible}) in a water-free environment. The filter with the ashes was placed on the crucible and heated progressively on a hot plate starting at room temperature up to 500° C. for a period of time of at least 1 hour. The crucible was then transferred to a furnace and heated at 800° C. for 16 h. After being cooled at room temperature in a desiccator, the crucible was weighted again (W_{crucible+ash}) in a water-free environment. AIA (%)=[(W_{crucible+ash}−W_{crucible})/Wₛₐₘₚₗₑ]×100.
D50, D90, D10 and D[4,3]: The method of determining the particle size distributions is complying with method <429> of the United Stated Pharmacopeia (USP40), and is based on the ISO standard 13320-1. A sample powder is first poured inside a vibrating hopper to feed with a regular flow a Mastersizer 3000 (Malvern). Using an air disperser device, the powder particles were blown through a laser beam with an obscuration of the light between 1 and 15%, to reach a sufficient signal-to-noise ratio of detector and to avoid multiple scattering. The light scattered by particles at different angles is measured by a multi-element detector. The use of red and blue light, coupled to the Mie theory allows the calculation of the volumetric size distribution, where particles were considered as spheres and hence an equivalent sphere size was determined. From the obtained size distribution the cumulative volume fractions at 10, 50 and 90% were determined to give D10, D50 and D90, respectively. The median diameter D50 gives an idea of the particle size of the powder, while D10 and D90 allows to quantify finer and coarser particle sizes.
CIELAB L*, a* and b* represent the most complete colour space specified by the International Commission on Illumination (Commission Internationale d'Eclairage). It describes all the colours visible to the human eye and was created to serve as a device independent model to be used as a reference. The L* and b* values of a sample are obtained by placing the sample in a glass cell (filled about half) of a colorimeter. The used colorimeter was a Minolta CR400 Colorimeter.

Rheology Measurements
Rheological measurements were carried out using a MCR 301 controlled-stress rheometer (Anton Paar Physica) equipped with a Couette device. The rheometer was also equipped with a Peltier temperature controller. Before measurements, samples were covered by a thin layer of paraffin oil to avoid evaporation during measurements. Dynamic oscillatory or viscoelastic measurements were selected to evaluate the gelation kinetics and texturizing properties of each investigated composition. For these measurements, sample was poured onto the MCR 301 plate pre-heated at 80° C. and was subjected to a temperature sweep test (2° C./min) from 80° C. down to 10° C., followed by a time sweep experiment for 15 minutes at a frequency of 0.4 Hz to ensure that the system reached an equilibrium state (structural rearrangements). After that, sample was subjected to a frequency sweep, from 100 to 0.01 Hz at a constant shear strain in the linear viscoelastic region (LVE), fixed at 0.3%. To ensure that viscoelastic measurements were carried out in the LVE domain, strain sweep experiments were conducted from 0.01% to 100% at 0.4 Hz. In all these rheological experiments, each measurement was performed at least in duplicate, from new sample preparations. The storage modulus (G′) values collected from the mechanical spectra at 0.1 Hz and at 10° C. are used for the comparison of all investigated samples.

Determination of C₀:

Sample Preparation for Rheology Measurements:
Reconstituted skimmed milk was used as the aqueous medium. The skimmed milk in powdered form was provided by Isigny-Ste-Mère (Isigny, France). The skimmed milk was reconstituted by dissolving powdered skimmed milk at 10% w/w in ultrapure water (18.2 MΩ·cm resistivity) under stirring for 4 hours at room temperature. In particular, to prepare 1000 g of reconstituted skimmed milk, 108.66 g of skimmed milk powder (DS=92.03 wt %) were dissolved in 891.34 g of ultrapure water. Dispersions of various seaweed-based powders were prepared in variable proportions (0.1 to 1 w/w. dry matter basis) in reconstituted skimmed milk. The seaweed-based powders were weighed in the suitable final proportion. thoroughly mixed with 5 wt % sucrose (to promote the rehydration) and slowly dispersed in the reconstituted skimmed milk under magnetic stirring (500 rpm). Stirring was maintained for 30 minutes at room temperature. Subsequently. the sample was heated to 80° C. for about 30 minutes under stirring at 500 rpm and held at this temperature for an additional 3 minutes.

Measurements of Storage Modulus G′:
Rheological measurements were carried out using a MCR 302 controlled-stress rheometer (Anton Paar Physica) equipped with a 50 mm plate-and-plate geometry with both upper and lower surface crosshatched. The rheometer is also equipped with a Peltier temperature controller. The gap was fixed at 1 mm Before measurements. samples were covered by a thin layer of paraffin oil on the edge of the sample to avoid evaporation during measurements. Dynamic oscillatory or viscoelastic measurements were selected to evaluate the gelation kinetics and texturizing properties of each formulated system. For these measurements, the sample was poured onto the MCR 302 plate pre-heated at 80° C. and subjected to a temperature sweep test (2° C./min) from 80° C. down to 10° C., followed by a time sweep experiment for 15 minutes at a frequency of 0.4 Hz to ensure that the system reach an equilibrium state after this considered time at 10° C. due to reorganization (structural rearrangements). Subsequently, the sample was subjected to a frequency sweep from 100 to 0.01 Hz at a constant shear strain in the linear viscoelastic region (LVE) fixed at 0.2%. To ensure that viscoelastic measurements were carried out in the LVE domain, strain sweep experiments were conducted from 0.01% to 100% at 0.4 Hz.
In all these rheological experiments. each measurement was performed at least in duplicate.

Data Processing: G′
The G′ values considered in this patent were collected from the mechanical spectra (frequency sweep test) at 0.4 Hz at 10° C. In fact as the mechanical spectra represents the real structural behaviour of the obtained gels, it appeared suitable to use this G′ value as the most appropriate parameter.
Based on the G′ values obtained for all investigated samples at various concentrations, a power-law relationship (see Formula 1) was used to describe the data. Note that c* represents the lowest concentration below which there is no gel-like behavior or implicitly the critical gelling concentration. C is the seaweed-based powder concentration (dry matter basis); n represents the exponent value of the fitting model; k and k′ are constant factors of the fitting model

G′=k′*(C−C₀)ⁿ Formula 1
To compare samples, Formulas 2-4 were used:

G′=p*k*Cⁿ Formula 2

G′_{sample A}=k*Cⁿ Formula 3

G′_{sample B}=p*k*Cⁿ Formula 4
where p is a translational shifting factor. If p=1, that means sample A displays similar gel strength as sample B; if p>1. that means sample B displays higher G′ than Sample A; if p<1 that means sample B displays lower G′ than Sample A.

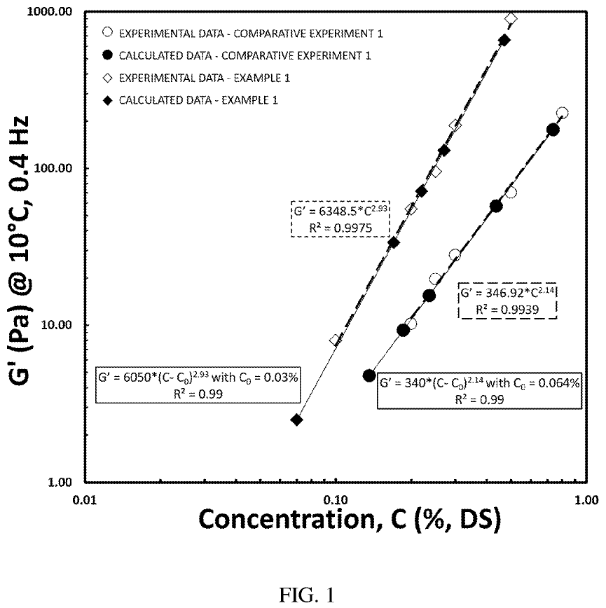

Data Processing: C₀
For the determination of C₀, the following steps were respected:
(i) The storage modulus G′ values collected from the mechanical spectra as described above were plotted as a function of seaweed-based powder concentration, C (%, DS), in logarithmic scales (see FIG. 1).
In FIG. 1, the dashed lines and the solid lines represent the fitting of the power law formulas 3 and 1, respectively, to the experimental data (raw data) and to the estimated data. The data utilized in FIG. 1, belongs to Example 1 and Comparative Example 1, respectively.
(ii) Following the approach described in literature (e.g. Agoda-Tandjawa, G., Dieudé-Fauvel, E., Girault, R. & Baudez, J.-C. (2013). Chemical Engineering Journal, 228, 799-805) equation G′=kCⁿ was mathematically transformed in the form G′=k′(C−C₀)ⁿ using linear regression. In this second equation, k′ represents the scaling factor, and C₀ the concentration below which no gel-like behaviour can be achieved. Note that the linear regression was performed for all investigated seaweed-based powders following the condition G′=kCⁿ=k′(C−C₀)ⁿ, with both exponents (n) values being identical and C>C₀.
The validation of C₀ determined using the above fitting model was verified by evaluating the rheological behaviour of all seaweed-based powders in similar conditions as described previously in other to evidence the gel-like behaviour.

The invention will now be described with the help of the following examples and comparative experiments, without being however limited thereto.

### Example 1: Producing a Kappaphycus alvarezii Powder

Fresh harvested (less than 6 h from the harvest) Kappaphycus alvarezii (Eucheuma cottonii) seaweed was rinsed with seawater and used to make a biomass having a DS of about 10 wt %. Seawater from the location of the harvest was used. The biomass was placed on a wooden table to form a biomass bed having an areal density of about 10 Kg/m². The table was placed in a sunny location and covered with a transparent tarpaulin to fully enclose it and prevent air flow. Due to the action of the sun, the temperature under the tarpaulin reached about 60° C. and a humidity over 90%. The seaweed was allowed to naturally exude in this environment for a period of time between 24 h and 72 h depending on the weather. After exudation, the tarpaulin was removed and the biomass was kept for another 24 h in open air under the sun for drying to reach a DS of about 78 wt %. The dried biomass was subsequently placed in volume of tap water sufficient to cover the seaweed entirely and the seaweed was allowed to rehydrate for 1 h at room temperature without stirring The rehydrated seaweed was then collected using a filter and a biomass having a DS around 40 wt % was obtained. The biomass containing the rehydrated seaweed was cooked in brine solution (100 g/L of KCl) at 90° C. for 30 minutes. The weight of the brine solution used for cooking was about 6 times the mass of the seaweed. After cooking, the brine solution was drained and the recovered seaweed was washed twice by placing it in a volume of tap water at room temperature for 10 minutes. Enough water was used to completely cover the seaweed. The seaweed was then collected using a filter and dried using a belt dryer for 30 minutes at 60° C. and led to a final product of about 94.9% DS. The dried product was milled into a powder with a Retsch mill (final sieve at 0.25 mm). The properties of the obtained seaweed powder are given in Table 1:

TABLE 1

| | | Seaweed |
|---|---|---|
| Property | | powder |
| Chloride (Cl⁻) (%) | | 4.4 |
| AIM (%) | | 11.2 |
| AIA (%) | | 0.3 |
| Color | L* | 78.4 |
| | a* | 1.3 |
| | b* | 14.2 |
| Particle | D50 (μm) | 87 |
| size | D90 (μm) | 228 |
| | D10 (μm) | 16 |
| | D [4,3] | 107 |
| | Span | 2.432 |

| | |
|---|---|
| G′ (Pa) | 130 |

### Example 2: Producing a Chondrus crispus Powder

A fresh Chondrus crispus was harvested from the wild. It was processed like in Example 1 and was kept between 3 and 72 hours under a tarpaulin. In some instances, the seaweed was turned over during the exudation to allow a homogeneous exposure to sunlight. The seaweed was then sun dried over a period ranging from 1 to 3.5 days, depending on the weather, to reach a DS of about 65 wt % (approximatively 35 wt % moisture). The seaweed was further processed as in Example 1.

The dried biomass was subsequently placed in volume of tap water sufficient to cover the seaweed entirely and the seaweed was allowed to rehydrate for 1 h at room temperature without stirring. The rehydrated seaweed was then collected using a filter and a biomass having a DS around 40 wt % was obtained.

The biomass containing the rehydrated seaweed was cooked twice in brine solution (350 g/L of KCl) at 90° C. for 30 minutes. The weight of the brine solution used for cooking was about 16 times the mass of the seaweed. After cooking, the brine solution was drained and the recovered seaweed was washed by placing it in a volume of tap water at room temperature for 10 minutes. Enough water was used to completely cover the seaweed.

The seaweed was then collected using a filter and dried using a belt dryer for 30 minutes at 60° C. and led to a final product of about 94.3% DS. The dried product was milled into a powder with a Retsch mill (final sieve at 0.25 mm) and sieved at 0.25 mm. The properties of the obtained seaweed powder are given in Table 2:

TABLE 2

| | | Seaweed-based |
|---|---|---|
| Property | | powder |
| Chloride (Cl⁻) (%) | | 0.1 |
| AIM (%) | | 8.1 |
| AIA (%) | | 0.1 |
| Color | L* | 58.9 |
| | a* | 5.3 |
| | b* | 8.9 |
| Particle | D50 (μm) | 164 |
| size | D90 (μm) | 310 |
| | D10 (μm) | 29 |
| | D [4,3] | 171 |
| | Span | 1.710 |

| | |
|---|---|
| G′ (Pa) | 140 |

### Example 3: Producing a Eucheuma spinosum Powder

Example 1 was repeated with the difference that the seaweed was Eucheuma spinosum, the brine solution contained 250 g/L KCl and the cooked biomass was washed three times in water. The properties of the obtained seaweed-based powder are given in Table 3:

TABLE 3

| | | Seaweed-based |
|---|---|---|
| Property | | powder |
| Chloride (Cl⁻) (%) | | 10.0 |
| AIM (%) | | 8.7 |
| AIA (%) | | 0.0 |
| Color | L* | 81.4 |
| | a* | 1.5 |
| | b* | 15.3 |
| Particle | D50 (μm) | 134 |
| size | D90 (μm) | 301 |
| | D10 (μm) | 21 |
| | D [4,3] | 149 |
| | Span | 2.089 |

| | |
|---|---|
| G′ (Pa) | 40 |

### Example 4: Dispersing the Seaweed Powders in Aqueous Environments

Standardized tap water was prepared by dispersing 6.85 g NaCl and 0.15 g CaCl₂.2H₂O in 1 L reverse osmosis water (17.1 mM NaCl and 1 mM CaCl₂) under stirring at room temperature.

Dispersions of the seaweed powders of Examples 1-3 were prepared at 0.5% DS in the standardized tap water from pH 3.5 pH to pH 7.0, according to the following method:
(i) The seaweed-based powders were weighted in the suitable final concentration and thoroughly dispersed in the standardized tap water under magnetic stirring at 500 rpm for 30 min
(ii) While dispersing, the pH of the dispersion was adjusted to the required value (e.g. 4.0) by using NaOH and HCl solutions (0.001-0.1N);
(iii) Subsequently, the sample was heated to 85° C. for about 30 minutes under stirring at 500 rpm and held at this temperature for an additional 3 minutes

Rheological measurements on the dispersions were carried out using a DHR3 controlled-stress rheometer (Discovery Hybrid Rheometer 3, TA. Instruments) equipped with a 40 mm plate-and-plate geometry with both upper and lower surface crosshatched. The rheometer was also equipped with a Peltier temperature controller. The gap was fixed at 1 mm Before measurements, samples were covered by a thin layer of paraffin oil on the edge of the sample to avoid evaporation during measurements.

Dynamic oscillatory or viscoelastic measurements were selected to evaluate the gelation kinetics and texturizing properties of each dispersion. For these measurements, the sample was poured onto the DHR3 plate pre-heated at 85° C. and subjected to a double heating and cooling down treatment from 85° C. to 10° C. with a holding time at 85° C. from 0 to 30 min (kinetic of 2° C./min under a constant shear strain of 0.2% in the viscoelastic range) as followed:

1) Temperature sweep test from 85° C. to 10° C. at 0.4 Hz

2) Time sweep experiment at 10° C. for 15 min at 0.4 Hz

3) Frequency sweep test at 10° C. from 100 Hz to 0.01 Hz

4) Temperature sweep test from 10° C. to 85° C. at 0.4 Hz

5) Time sweep experiment at 85° C. from 0 min to 30 min at 0.4 Hz

6) Temperature sweep test from 85° C. to 10° C. at 0.4 Hz

7) Time sweep experiment at 10° C. for 15 min at 0.4 Hz

8) Frequency sweep test at 10° C. from 100 Hz to 0.01 Hz

### Results

The inventors observed that all dispersions of the seaweed powder in water exhibited a true gel-like behaviour in the aqueous environment with G′>10 G″ (Table 4). Gelation temperatures reveal that the lower the pH and the higher the holding time at 85° C., the higher the gelation delay (globally more than 10° C.). Additionally, the functionality of the seaweed powders remains relatively stable when dispersed in accordance with the invention, in a wide range of pH (e.g. 4-7).

TABLE 4

| | heating | Powder of Example 1 | | | Powder of Example 3 | | |
|---|---|---|---|---|---|---|---|
| | time at | G′ | | Gelation | G′ | | Gelation |
| pH | 85° C. | (Pa) | Tan δ | temp. | (Pa) | Tan δ | temp. |
| pH 3.0 | 0 | 1.3 | 0.425 | | | | |
| | 20 | 0.1 | 0.595 | | | | |
| | 30 | 1.7 | 0.227 | | | | |
| pH 3.5 | 0 | 16.4 | 0.04 | | | | |
| | 30 | 4.8 | 0.072 | 10 | 10.4 | 0.045 | 18 |
| pH 4.0 | 0 | 72.6 | 0.072 | 14 | | | |
| | 30 | 68.4 | 0.026 | 14 | | | |
| pH 4.5 | 0 | 83.8 | 0.032 | 16 | | | |
| | 30 | 82.0 | 0.031 | 16 | | | |
| pH 6.0 | 0 | 81.4 | 0.029 | 16 | | | |
| | 30 | 78.3 | 0.030 | 16 | | | |
| pH 7.0 | 0 | 80.0 | 0.03 | 16 | 17.7 | 0.028 | 32 |
| | 3.0 | 80.9 | 0.029 | 16 | 17.1 | 0.030 | 33 |

1. A method of dispersing a seaweed powder in an aqueous environment, comprising the steps of:
a. providing a seaweed powder and an aqueous environment; the seaweed powder having a storage modulus (G′) of at least 10 Pa as determined on a 0.3 wt % aqueous dispersion of said powder;
b. dispersing the seaweed powder in the aqueous environment at a pH of at least 3.5, preferably at most 9.0.

2. The method of claim 1, wherein the seaweed powder contains seaweed particles having a D50 of at least 20 μm.

3. The method of claim 1, wherein the seaweed powder contains seaweed particles having a D90 of at least 125 μm.

4. The method of claim 1, wherein the seaweed is a red, a brown or a green seaweed.

5. The method of claim 1, wherein the seaweed is a red seaweed selected from the families of Gigartinaceae, Bangiophyceae, Palmariaceae, Hypneaceae, Cystocloniaceae, Solieriaceae, Phyllophoraceae and Furcellariaceae or combinations thereof.

6. The method of claim 1, wherein the seaweed powder has a critical gelling concentration (C0) of at most 0.5 wt %.

7. The method of claim 1, wherein the seaweed powder contains an amount of acid insoluble ashes (AIA) of at most 5.0 wt % relative to the weight of the powder.

8. The method of claim 1, wherein the seaweed powder contains an amount of acid insoluble material (AIM) of at most 50 wt % relative to the weight of the powder.

9. The method of claim 1, wherein the aqueous environment contains at least 30 wt % water based on the total weight of said environment.

10. The method of claim 1, wherein the aqueous environment contains a salt.

11. The method of claim 1, wherein the aqueous environment has an ionic strength of at least 0.01 M.

12. The method of claim 1, wherein the seaweed powder is dispersed in the aqueous environment in an amount of at least 0.1 wt % based on the total dry solids content of said environment.

13. A dispersion of a seaweed in an aqueous environment, said dispersion having a pH of at least 3.5.

14. The dispersion of claim 13, having an ionic strength of at least 0.01M.
